(19) United States
(12) Patent Application Publication
(10) Pub. No.: US 2021/0290066 A1
(43) Pub. Date: Sep. 23, 2021

(54) Dynamic Range Optimization in an Optical Measurement System

(71) Applicant: HI LLC, Los Angeles, CA (US)

(72) Inventors: Ryan Field, Culver City, CA (US); Bruno Do Valle, Brighton, MA (US); Jacob Dahle, Arlington, MA (US)

(21) Appl. No.: 17/202,651

(22) Filed: Mar. 16, 2021

Related U.S. Application Data
(60) Provisional application No. 62/992,526, filed on Mar. 20, 2020.
(60) Provisional application No. 63/057,077, filed on Jul. 27, 2020.

(51) Int. Cl. A61B 5/00; G01J 1/42; G01J 1/44; G04F 10/00

(52) U.S. Cl. CPC A61B 5/0075; G01J 1/4228; G01J 1/44; G04F 10/005; G01J 2001/442; A61B 2562/0238; G01J 2001/4466; G01J 2001/448; A61B 5/0042

(57) ABSTRACT

An illustrative optical measurement system includes a light source configured to emit, during a measurement session time period, a sequence of light pulses towards a target; a plurality of photodetectors configured to remain in an armed state during an entire duration of the measurement session time period, and detect, while in the armed state, photons of the light pulses after the light pulses are scattered by the target; and a plurality of time-to-digital converters (TDCs) comprising a different TDC for each photodetector of the plurality of photodetectors, the TDCs configured to record timestamp symbols representative of when the photons are detected by the photodetectors.

### RELATED APPLICATIONS

The present application claims priority under 35 U.S.C. § 119(e) to U.S. Provisional Patent Application No. 62/992,526, filed on Mar. 20, 2020, and to U.S. Provisional Patent Application No. 63/057,077, filed on Jul. 27, 2020. These applications are incorporated herein by reference in their respective entireties.

### BACKGROUND INFORMATION

Detecting neural activity in the brain (or any other turbid medium) is useful for medical diagnostics, imaging, neuroengineering, brain-computer interfacing, and a variety of other diagnostic and consumer-related applications. For example, it may be desirable to detect neural activity in the brain of a user to determine if a particular region of the brain has been impacted by reduced blood irrigation, a hemorrhage, or any other type of damage. As another example, it may be desirable to detect neural activity in the brain of a user and computationally decode the detected neural activity into commands that can be used to control various types of consumer electronics (e.g., by controlling a cursor on a computer screen, changing channels on a television, turning lights on, etc.).

Neural activity and other attributes of the brain may be determined or inferred by measuring responses of tissue within the brain to light pulses. One technique to measure such responses is time-correlated single-photon counting (TCSPC). Time-correlated single-photon counting detects single photons and measures a time of arrival of the photons with respect to a reference signal (e.g., a light source). By repeating the light pulses, TCSPC may accumulate a sufficient number of photon events to statistically determine a histogram representing the distribution of detected photons. Based on the histogram of photon distribution, the response of tissue to light pulses may be determined in order to study the detected neural activity and/or other attributes of the brain.

A photodetector capable of detecting a single photon (i.e., a single particle of optical energy) is an example of a non-invasive detector that can be used in an optical measurement system to detect neural activity within the brain. An exemplary photodetector is implemented by a semiconductor-based single-photon avalanche diode (SPAD), which is capable of capturing individual photons with very high time-of-arrival resolution (a few tens of picoseconds).

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an exemplary optical measurement system.

FIG. 2 illustrates an exemplary detector architecture.

FIG. 3 illustrates an exemplary timing diagram for performing an optical measurement operation using an optical measurement system.

FIG. 4 illustrates a graph of an exemplary temporal point spread function that may be generated by an optical measurement system in response to a light pulse.

FIG. 5 shows an exemplary non-invasive wearable brain interface system.

FIG. 6 shows an exemplary optical measurement system.

FIG. 7 shows an illustrative modular assembly.

FIGS. 8A-8B show an exemplary implementation of the modular assembly of FIG. 7.

FIG. 9 shows exemplary histograms.

FIG. 10 shows an exemplary optical measurement system.

FIG. 11 illustrates an exemplary timing diagram.

FIG. 12 shows an exemplary implementation of the optical measurement system of FIG. 10.

FIG. 13 illustrates an exemplary implementation of a processing unit.

FIG. 14 shows an exemplary configuration in which a processing unit is configured to generate a single histogram based on output signals provided by time-to-digital converters of multiple detectors of a module.

FIG. 15 shows an exemplary configuration in which a processing unit is configured to generate multiple histograms each based on a different subset of output signals provided by time-to-digital converters of multiple detectors of a module.

FIGS. 16-21 illustrate embodiments of a wearable device that includes elements of the optical detection systems described herein.

FIG. 22 illustrates an exemplary computing device.

### DETAILED DESCRIPTION

In accordance with the systems, circuits, and methods described herein, an optical measurement system may include parallel arrays of photodetectors and time-to-digital converters (TDC). In other words, each TDC included in the array of TDCs corresponds to a different photodetector included in the array of photodetectors. As described herein, this parallelism may provide increased dynamic range compared to conventional optical measurement systems (e.g., silicon photomultiplier (SiPM) systems) that include only one TDC for a plurality of photodetectors.

Moreover, the systems, circuits, and methods described herein may mitigate the effect of photon pile-up that may occur when relatively high intensity light (e.g., in terms of count rates, which are typically measured by percent of light pulses for which a photon is detected) is used to perform optical measurements. As such, the systems, circuits, and methods may allow for the use of relatively high intensity light (e.g., that supports relatively high count rates, such as ten percent or more), which may facilitate measurement times that are fast enough to capture signals of interest (e.g., hemodynamic signals).

As described herein, the photodetector arrays and TDC arrays described herein may include a relatively high number (e.g., 256, 512, . . . , 16384, etc.) of individual photodetectors and TDCs, respectively. As such, even at relatively high excitation rates (e.g., photon detections in response to more than one out of 10 light pulses), there will still be a large number of photodetectors available to observe late arriving photons. As described herein, this may obviate the need for fast gating circuitry for each of the photodetectors, which may conserve power and allow for the design of relatively small devices (e.g., the housing components containing the electronics embedded within a wearable optical measurement system as described herein).

These and other advantages and benefits of the present systems, circuits, and methods are described more fully herein.

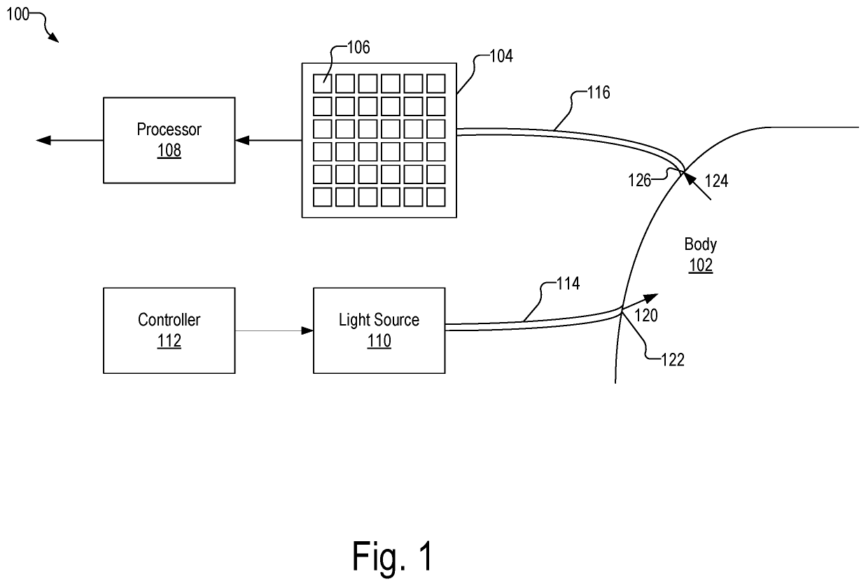

FIG. 1 shows an exemplary optical measurement system 100 configured to perform an optical measurement operation with respect to a body 102. Optical measurement system 100 may, in some examples, be portable and/or wearable by a user. Optical measurement systems that may be used in connection with the embodiments described herein are described more fully in U.S. patent application Ser. No. 17/176,315, filed Feb. 16, 2021; U.S. patent application Ser. No. 17/176,309, filed Feb. 16, 2021; U.S. patent application Ser. No. 17/176,460, filed Feb. 16, 2021; U.S. patent application Ser. No. 17/176,470, filed Feb. 16, 2021; U.S. patent application Ser. No. 17/176,487, filed Feb. 16, 2021; U.S. patent application Ser. No. 17/176,539, filed Feb. 16, 2021; U.S. patent application Ser. No. 17/176,560, filed Feb. 16, 2021; and U.S. patent application Ser. No. 17/176,466, filed Feb. 16, 2021, which applications are incorporated herein by reference in their entirety.

In some examples, optical measurement operations performed by optical measurement system 100 are associated with a time domain-based optical measurement technique. Example time domain-based optical measurement techniques include, but are not limited to, TCSPC, time domain near infrared spectroscopy (TD-NIRS), time domain diffusive correlation spectroscopy (TD-DCS), and time domain Digital Optical Tomography (TD-DOT).

As shown, optical measurement system 100 includes a detector 104 that includes a plurality of individual photodetectors (e.g., photodetector 106), a processor 108 coupled to detector 104, a light source 110, a controller 112, and optical conduits 114 and 116 (e.g., light pipes). However, one or more of these components may not, in certain embodiments, be considered to be a part of optical measurement system 100. For example, in implementations where optical measurement system 100 is wearable by a user, processor 108 and/or controller 112 may in some embodiments be separate from optical measurement system 100 and not configured to be worn by the user.

Detector 104 may include any number of photodetectors 106 as may serve a particular implementation, such as 2ⁿ photodetectors (e.g., 256, 512, . . . , 16384, etc.), where n is an integer greater than or equal to one (e.g., 4, 5, 8, 10, 11, 14, etc.). Photodetectors 106 may be arranged in any suitable manner.

Photodetectors 106 may each be implemented by any suitable circuit configured to detect individual photons of light incident upon photodetectors 106. For example, each photodetector 106 may be implemented by a single photon avalanche diode (SPAD) circuit and/or other circuitry as may serve a particular implementation.

Processor 108 may be implemented by one or more physical processing (e.g., computing) devices. In some examples, processor 108 may execute instructions (e.g., software) configured to perform one or more of the operations described herein.

Light source 110 may be implemented by any suitable component configured to generate and emit light. For example, light source 110 may be implemented by one or more laser diodes, distributed feedback (DFB) lasers, super luminescent diodes (SLDs), light emitting diodes (LEDs), diode-pumped solid-state (DPSS) lasers, super luminescent light emitting diodes (sLEDs), vertical-cavity surface-emitting lasers (VCSELs), titanium sapphire lasers, micro light emitting diodes (mLEDs), and/or any other suitable laser or light source. In some examples, the light emitted by light source 110 is high coherence light (e.g., light that has a coherence length of at least 5 centimeters) at a predetermined center wavelength.

Light source 110 is controlled by controller 112, which may be implemented by any suitable computing device (e.g., processor 108), integrated circuit, and/or combination of hardware and/or software as may serve a particular implementation. In some examples, controller 112 is configured to control light source 110 by turning light source 110 on and off and/or setting an intensity of light generated by light source 110. Controller 112 may be manually operated by a user, or may be programmed to control light source 110 automatically.

Light emitted by light source 110 may travel via an optical conduit 114 (e.g., a light pipe, a light guide, a waveguide, a single-mode optical fiber, and/or or a multi-mode optical fiber) to body 102 of a subject. In cases where optical conduit 114 is implemented by a light guide, the light guide may be spring loaded and/or have a cantilever mechanism to allow for conformably pressing the light guide firmly against body 102.

Body 102 may include any suitable turbid medium. For example, in some implementations, body 102 is a head or any other body part of a human or other animal. Alternatively, body 102 may be a non-living object. For illustrative purposes, it will be assumed in the examples provided herein that body 102 is a human head.

As indicated by arrow 120, the light emitted by light source 110 enters body 102 at a first location 122 on body 102. Accordingly, a distal end of optical conduit 114 may be positioned at (e.g., right above, in physical contact with, or physically attached to) first location 122 (e.g., to a scalp of the subject). In some examples, the light may emerge from optical conduit 114 and spread out to a certain spot size on body 102 to fall under a predetermined safety limit. At least a portion of the light as indicated by arrow 120 may be scattered within body 102.

As used herein, “distal” means nearer, along the optical path of the light emitted by light source 110 or the light received by detector 104, to the target (e.g., within body 102) than to light source 110 or detector 104. Thus, the distal end of optical conduit 114 is nearer to body 102 than to light source 110, and the distal end of optical conduit 116 is nearer to body 102 than to detector 104. Additionally, as used herein, “proximal” means nearer, along the optical path of the light emitted by light source 110 or the light received by detector 104, to light source 110 or detector 104 than to body 102. Thus, the proximal end of optical conduit 114 is nearer to light source 110 than to body 102, and the proximal end of optical conduit 116 is nearer to detector 104 than to body 102.

As shown, the distal end of optical conduit 116 (e.g., a light pipe, a light guide, a waveguide, a single-mode optical fiber, and/or a multi-mode optical fiber) is positioned at (e.g., right above, in physical contact with, or physically attached to) output location 126 on body 102. In this manner, optical conduit 116 may collect at least a portion of the scattered light (indicated as light 124) as it exits body 102 at location 126 and carry light 124 to detector 104. Light 124 may pass through one or more lenses and/or other optical elements (not shown) that direct light 124 onto each of the photodetectors 106 included in detector 104.

Photodetectors 106 may be connected in parallel in detector 104. An output of each of photodetectors 106 may be accumulated to generate an accumulated output of detector 104. Processor 108 may receive the accumulated output and determine, based on the accumulated output, a temporal distribution of photons detected by photodetectors 106. Processor 108 may then generate, based on the temporal distribution, a histogram representing a light pulse response of a target (e.g., brain tissue, blood flow, etc.) in body 102. Example embodiments of accumulated outputs are described herein.

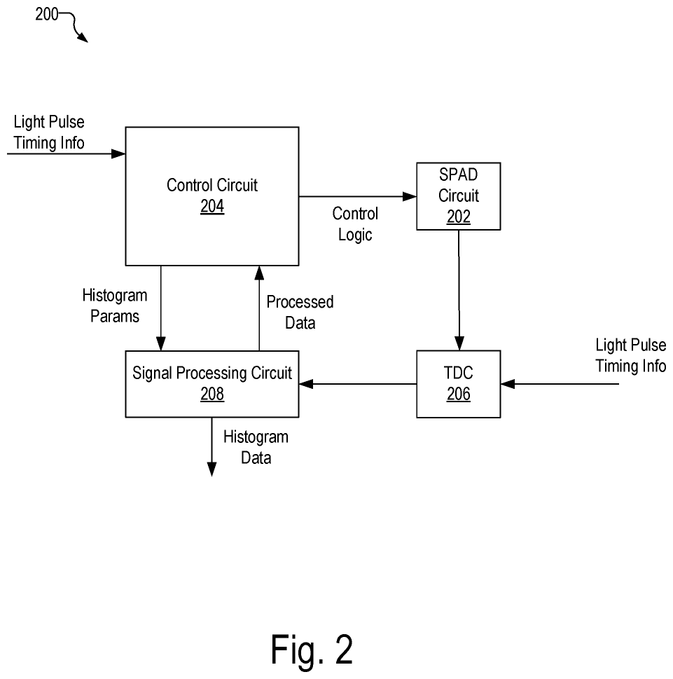

FIG. 2 illustrates an exemplary detector architecture 200 that may be used in accordance with the systems and methods described herein. As shown, architecture 200 includes a SPAD circuit 202 that implements photodetector 106, a control circuit 204, a time-to-digital converter (TDC) 206, and a signal processing circuit 208. Architecture 200 may include additional or alternative components as may serve a particular implementation.

In some examples, SPAD circuit 202 may include a SPAD and a fast gating circuit configured to operate together to detect a photon incident upon the SPAD. As described herein, SPAD circuit 202 may generate an output when SPAD circuit 202 detects a photon.

The fast gating circuit included in SPAD circuit 202 may be implemented in any suitable manner. For example, the fast gating circuit may be implemented by an active voltage source, a capacitor that is pre-charged with a bias voltage before a command is provided to arm the SPAD, and/or in any other suitable manner.

In some alternative configurations, SPAD circuit 202 does not include a fast gating circuit. In these configurations, the SPAD included in SPAD circuit 202 may be gated in any suitable manner or be configured to operate in a free running mode with passive quenching.

Control circuit 204 may be implemented by an application specific integrated circuit (ASIC) or any other suitable circuit configured to control an operation of various components within SPAD circuit 202. For example, control circuit 204 may output control logic that puts the SPAD included in SPAD circuit 202 in either an armed or a disarmed state.

In some examples, control circuit 204 may control an arming and a disarming of a SPAD included in SPAD circuit 202. Control circuit 204 may also control a programmable gate width, which specifies how long the SPAD is kept in an armed state before being disarmed.

Control circuit 204 is further configured to control signal processing circuit 208. For example, control circuit 204 may provide histogram parameters (e.g., time bins, number of light pulses, type of histogram, etc.) to signal processing circuit 208. Signal processing circuit 208 may generate histogram data in accordance with the histogram parameters. In some examples, control circuit 204 is at least partially implemented by controller 112.

TDC 206 is configured to measure a time difference between an occurrence of an output pulse generated by SPAD circuit 202 and an occurrence of a light pulse. To this end, TDC 206 may also receive the same light pulse timing information that control circuit 204 receives. TDC 206 may be implemented by any suitable circuitry as may serve a particular implementation.

Signal processing circuit 208 is configured to perform one or more signal processing operations on data output by TDC 206. For example, signal processing circuit 208 may generate histogram data based on the data output by TDC 206 and in accordance with histogram parameters provided by control circuit 204. To illustrate, signal processing circuit 208 may generate, store, transmit, compress, analyze, decode, and/or otherwise process histograms based on the data output by TDC 206. In some examples, signal processing circuit 208 may provide processed data to control circuit 204, which may use the processed data in any suitable manner. In some examples, signal processing circuit 208 is at least partially implemented by processor 108.

In some examples, each photodetector 106 (e.g., SPAD circuit 202) may have a dedicated TDC 206 associated therewith. For example, for an array of N photodetectors 106, there may be a corresponding array of N TDCs 206. Likewise, a single control circuit 204 and a single signal processing circuit 208 may be provided for a one or more photodetectors 106 and/or TDCs 206.

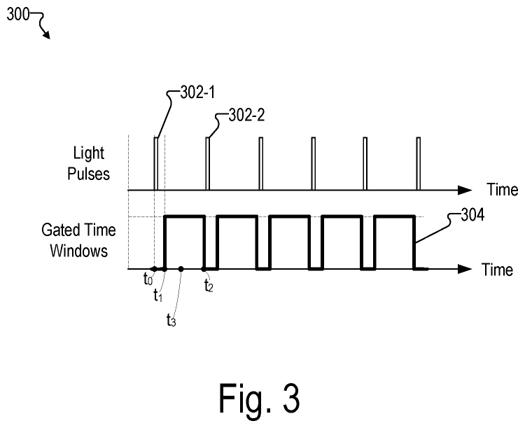

FIG. 3 illustrates an exemplary timing diagram 300 for performing an optical measurement operation using optical measurement system 100. Optical measurement system 100 may be configured to perform the optical measurement operation by directing light pulses (e.g., laser pulses) toward a target within a body (e.g., body 102). The light pulses may be short (e.g., 10-2000 picoseconds (ps)) and repeated at a high frequency (e.g., between 100,000 hertz (Hz) and 100 megahertz (MHz)). The light pulses may be scattered by the target and then detected by optical measurement system 100. Optical measurement system 100 may measure a time relative to the light pulse for each detected photon. By counting the number of photons detected at each time relative to each light pulse repeated over a plurality of light pulses, optical measurement system 100 may generate a histogram that represents a light pulse response of the target (e.g., a temporal point spread function (TPSF)). The terms histogram and TPSF are used interchangeably herein to refer to a light pulse response of a target.

For example, timing diagram 300 shows a sequence of light pulses 302 (e.g., light pulses 302-1 and 302-2) that may be applied to the target (e.g., tissue within a brain of a user, blood flow, a fluorescent material used as a probe in a body of a user, etc.). Timing diagram 300 also shows a pulse wave 304 representing predetermined gated time windows (also referred as gated time periods) during which photodetectors 106 are gated ON (i.e., armed) to detect photons. Referring to light pulse 302-1, light pulse 302-1 is applied at a time t₀. At a time t₁, a first instance of the predetermined gated time window begins. Photodetectors 106 may be armed at time t₁, enabling photodetectors 106 to detect photons scattered by the target during the predetermined gated time window. In this example, time t₁ is set to be at a certain time after time t₀, which may minimize photons detected directly from the laser pulse, before the laser pulse reaches the target. However, in some alternative examples, time t₁ is set to be equal to time t₀.

At a time t₂, the predetermined gated time window ends. In some examples, photodetectors 106 may be disarmed at time t₂. In other examples, photodetectors 106 may be reset (e.g., disarmed and re-armed) at time t₂ or at a time subsequent to time t₂. During the predetermined gated time window, photodetectors 106 may detect photons scattered by the target. Photodetectors 106 may be configured to remain armed during the predetermined gated time window such that photodetectors 106 maintain an output upon detecting a photon during the predetermined gated time window. For example, a photodetector 106 may detect a photon at a time t₃, which is during the predetermined gated time window between times t₁ and t₂. The photodetector 106 may be configured to provide an output indicating that the photodetector 106 has detected a photon. The photodetector 106 may be configured to continue providing the output until time t₂, when the photodetector may be disarmed and/or reset. Optical measurement system 100 may generate an accumulated output from the plurality of photodetectors. Optical measurement system 100 may sample the accumulated output to determine times at which photons are detected by photodetectors 106 to generate a TPSF.

As described herein, the systems, circuits, and methods described herein may obviate the need for the gated time windows described in connection with FIG. 3, thereby obviating the need for fast gating circuitry to be included in optical measurement system 100.

As mentioned, in some alternative examples, photodetector 106 may be configured to operate in a free-running mode such that photodetector 106 is not actively armed and disarmed (e.g., at the end of each predetermined gated time window represented by pulse wave 304). In contrast, while operating in the free-running mode, photodetector 106 may be configured to reset within a configurable time period after an occurrence of a photon detection event (i.e., after photodetector 106 detects a photon) and immediately begin detecting new photons. However, only photons detected within a desired time window (e.g., during each gated time window represented by pulse wave 304) may be included in the TPSF.

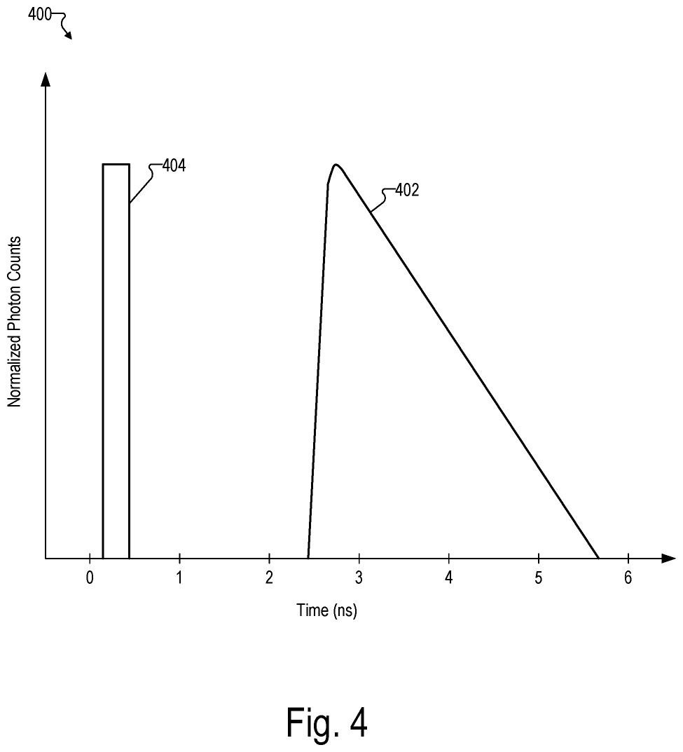

FIG. 4 illustrates a graph 400 of an exemplary TPSF 402 that may be generated by optical measurement system 100 in response to a light pulse 404 (which, in practice, represents a plurality of light pulses). Graph 400 shows a normalized count of photons on a y-axis and time bins on an x-axis. As shown, TPSF 402 is delayed with respect to a temporal occurrence of light pulse 404. In some examples, the number of photons detected in each time bin subsequent to each occurrence of light pulse 404 may be aggregated (e.g., integrated) to generate TPSF 402. TPSF 402 may be analyzed and/or processed in any suitable manner to determine or infer detected neural activity.

Optical measurement system 100 may be implemented by or included in any suitable device. For example, optical measurement system 100 may be included, in whole or in part, in a non-invasive wearable device (e.g., a headpiece) that a user may wear to perform one or more diagnostic, imaging, analytical, and/or consumer-related operations. The non-invasive wearable device may be placed on a user's head or other part of the user to detect neural activity. In some examples, such neural activity may be used to make behavioral and mental state analysis, awareness and predictions for the user.

Mental state described herein refers to the measured neural activity related to physiological brain states and/or mental brain states, e.g., joy, excitement, relaxation, surprise, fear, stress, anxiety, sadness, anger, disgust, contempt, contentment, calmness, focus, attention, approval, creativity, positive or negative reflections/attitude on experiences or the use of objects, etc. Further details on the methods and systems related to a predicted brain state, behavior, preferences, or attitude of the user, and the creation, training, and use of neuromes can be found in U.S. Provisional Patent Application No. 63/047,991, filed Jul. 3, 2020. Exemplary measurement systems and methods using biofeedback for awareness and modulation of mental state are described in more detail in U.S. patent application Ser. No. 16/364,338, filed Mar. 26, 2019, published as US2020/0196932A1. Exemplary measurement systems and methods used for detecting and modulating the mental state of a user using entertainment selections, e.g., music, film/video, are described in more detail in U.S. patent application Ser. No. 16/835,972, filed Mar. 31, 2020, published as US2020/0315510A1. Exemplary measurement systems and methods used for detecting and modulating the mental state of a user using product formulation from, e.g., beverages, food, selective food/drink ingredients, fragrances, and assessment based on product-elicited brain state measurements are described in more detail in U.S. patent application Ser. No. 16/853,614, filed Apr. 20, 2020, published as US2020/0337624A1. Exemplary measurement systems and methods used for detecting and modulating the mental state of a user through awareness of priming effects are described in more detail in U.S. patent application Ser. No. 16/885,596, filed May 28, 2020, published as US2020/0390358A1. These applications and corresponding U.S. publications are incorporated herein by reference in their entirety.

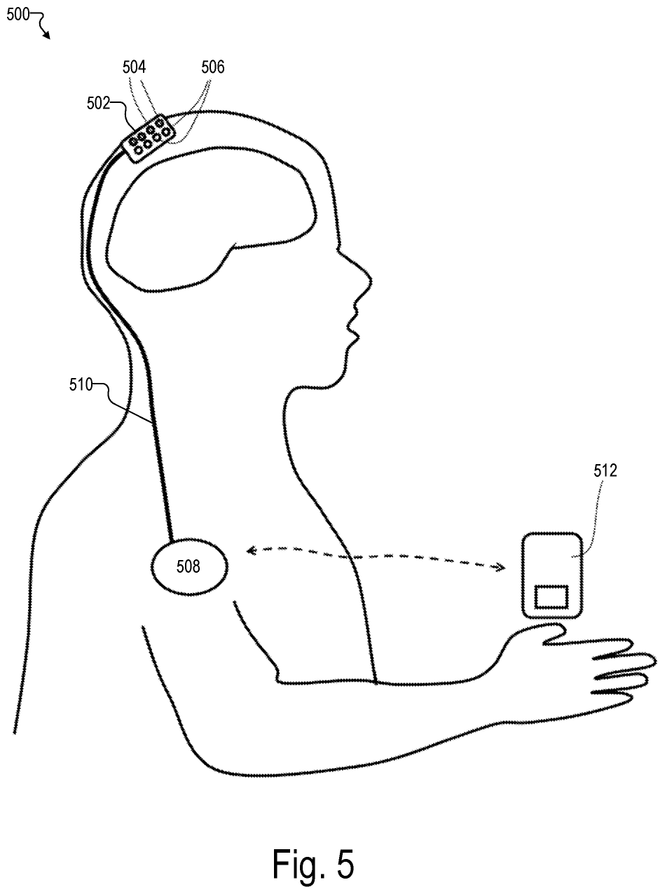

FIG. 5 shows an exemplary non-invasive wearable brain interface system 500 (“brain interface system 500”) that implements optical measurement system 100 (shown in FIG. 1). As shown, brain interface system 500 includes a head-mountable component 502 configured to be attached to a user's head. Head-mountable component 502 may be implemented by a cap shape that is worn on a head of a user. Alternative implementations of head-mountable component 502 include helmets, beanies, headbands, other hat shapes, or other forms conformable to be worn on a user's head, etc. Head-mountable component 502 may be made out of any suitable cloth, soft polymer, plastic, hard shell, and/or any other suitable material as may serve a particular implementation. Examples of headgears used with wearable brain interface systems are described more fully in U.S. Pat. No. 10,340,408, incorporated herein by reference in its entirety.

Head-mountable component 502 includes a plurality of detectors 504, which may implement or be similar to detector 104, and a plurality of light sources 506, which may be implemented by or be similar to light source 110. It will be recognized that in some alternative embodiments, head-mountable component 502 may include a single detector 504 and/or a single light source 506.

Brain interface system 500 may be used for controlling an optical path to the brain and for transforming photodetector measurements into an intensity value that represents an optical property of a target within the brain. Brain interface system 500 allows optical detection of deep anatomical locations beyond skin and bone (e.g., skull) by extracting data from photons originating from light source 506 and emitted to a target location within the user's brain, in contrast to conventional imaging systems and methods (e.g., optical coherence tomography (OCT)), which only image superficial tissue structures or through optically transparent structures.

Brain interface system 500 may further include a processor 508 configured to communicate with (e.g., control and/or receive signals from) detectors 504 and light sources 506 by way of a communication link 510. Communication link 510 may include any suitable wired and/or wireless communication link. Processor 508 may include any suitable housing and may be located on the user's scalp, neck, shoulders, chest, or arm, as may be desirable. In some variations, processor 508 may be integrated in the same assembly housing as detectors 504 and light sources 506.

As shown, brain interface system 500 may optionally include a remote processor 512 in communication with processor 508. For example, remote processor 512 may store measured data from detectors 504 and/or processor 508 from previous detection sessions and/or from multiple brain interface systems (not shown). Power for detectors 504, light sources 506, and/or processor 508 may be provided via a wearable battery (not shown). In some examples, processor 508 and the battery may be enclosed in a single housing, and wires carrying power signals from processor 508 and the battery may extend to detectors 504 and light sources 506. Alternatively, power may be provided wirelessly (e.g., by induction).

In some alternative embodiments, head mountable component 502 does not include individual light sources. Instead, a light source configured to generate the light that is detected by detector 504 may be included elsewhere in brain interface system 500. For example, a light source may be included in processor 508 and coupled to head mountable component 502 through optical connections.

Optical measurement system 100 may alternatively be included in a non-wearable device (e.g., a medical device and/or consumer device that is placed near the head or other body part of a user to perform one or more diagnostic, imaging, and/or consumer-related operations). Optical measurement system 100 may alternatively be included in a sub-assembly enclosure of a wearable invasive device (e.g., an implantable medical device for brain recording and imaging).

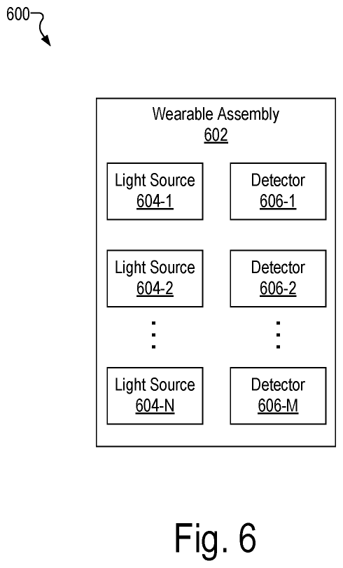

FIG. 6 shows an exemplary optical measurement system 600 in accordance with the principles described herein. Optical measurement system 600 may be an implementation of optical measurement system 100 and, as shown, includes a wearable assembly 602, which includes N light sources 604 (e.g., light sources 604-1 through 604-N) and M detectors 606 (e.g., detectors 606-1 through 606-M). Optical measurement system 600 may include any of the other components of optical measurement system 100 as may serve a particular implementation. N and M may each be any suitable value (i.e., there may be any number of light sources 604 and detectors 606 included in optical measurement system 600 as may serve a particular implementation).

Light sources 604 are each configured to emit light (e.g., a sequence of light pulses) and may be implemented by any of the light sources described herein. Detectors 606 may each be configured to detect arrival times for photons of the light emitted by one or more light sources 604 after the light is scattered by the target. For example, a detector 606 may include a photodetector configured to generate a photodetector output pulse in response to detecting a photon of the light and a TDC configured to record a timestamp symbol in response to an occurrence of the photodetector output pulse, the timestamp symbol representative of an arrival time for the photon (i.e., when the photon is detected by the photodetector).

Wearable assembly 602 may be implemented by any of the wearable devices, modular assemblies, and/or wearable units described herein. For example, wearable assembly 602 may be implemented by a wearable device (e.g., headgear) configured to be worn on a user's head. Wearable assembly 602 may additionally or alternatively be configured to be worn on any other part of a user's body.

Optical measurement system 600 may be modular in that one or more components of optical measurement system 600 may be removed, changed out, or otherwise modified as may serve a particular implementation. As such, optical measurement system 600 may be configured to conform to three-dimensional surface geometries, such as a user's head. Exemplary modular multimodal measurement systems are described in more detail in U.S. Provisional patent application Ser. No. 17/176,460, filed Feb. 16, 2021, U.S. Provisional patent application Ser. No. 17/176,470, filed Feb. 16, 2021, U.S. Provisional patent application Ser. No. 17/176,487, filed Feb. 16, 2021, U.S. Provisional Patent Application No. 63/038,481, filed Feb. 16, 2021, and U.S. Provisional patent application Ser. No. 17/176,560, filed Feb. 16, 2021, which applications are incorporated herein by reference in their respective entireties.

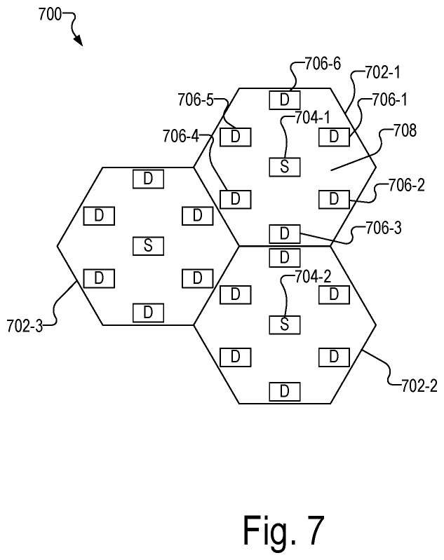

FIG. 7 shows an illustrative modular assembly 700 that may implement optical measurement system 600. Modular assembly 700 is illustrative of the many different implementations of optical measurement system 600 that may be realized in accordance with the principles described herein.

As shown, modular assembly 700 includes a plurality of modules 702 (e.g., modules 702-1 through 702-3). While three modules 702 are shown to be included in modular assembly 700, in alternative configurations, any number of modules 702 (e.g., a single module up to sixteen or more modules) may be included in modular assembly 700.

Each module 702 includes a light source (e.g., light source 704-1 of module 702-1 and light source 704-2 of module 702-2) and a plurality of detectors (e.g., detectors 706-1 through 706-6 of module 702-1). In the particular implementation shown in FIG. 7, each module 702 includes a single light source and six detectors. Each light source is labeled “S” and each detector is labeled “D”.

Each light source depicted in FIG. 7 may be implemented by one or more light sources similar to light source 110 and may be configured to emit light directed at a target (e.g., the brain).

Each light source depicted in FIG. 7 may be located at a center region of a surface of the light source's corresponding module. For example, light source 704-1 is located at a center region of a surface 708 of module 702-1. In alternative implementations, a light source of a module may be located away from a center region of the module.

Each detector depicted in FIG. 7 may implement or be similar to detector 104 and may include a plurality of photodetectors (e.g., SPADs) as well as other circuitry (e.g., TDCs), and may be configured to detect arrival times for photons of the light emitted by one or more light sources after the light is scattered by the target.

The detectors of a module may be distributed around the light source of the module. For example, detectors 706 of module 702-1 are distributed around light source 704-1 on surface 708 of module 702-1. In this configuration, detectors 706 may be configured to detect photon arrival times for photons included in light pulses emitted by light source 704-1. In some examples, one or more detectors 706 may be close enough to other light sources to detect photon arrival times for photons included in light pulses emitted by the other light sources. For example, because detector 706-3 is adjacent to module 702-2, detector 706-3 may be configured to detect photon arrival times for photons included in light pulses emitted by light source 704-2 (in addition to detecting photon arrival times for photons included in light pulses emitted by light source 704-1).

In some examples, the detectors of a module may all be equidistant from the light source of the same module. In other words, the spacing between a light source (i.e., a distal end portion of a light source optical conduit) and the detectors (i.e., distal end portions of optical conduits for each detector) are maintained at the same fixed distance on each module to ensure homogeneous coverage over specific areas and to facilitate processing of the detected signals. The fixed spacing also provides consistent spatial (lateral and depth) resolution across the target area of interest, e.g., brain tissue. Moreover, maintaining a known distance between the light source, e.g., light emitter, and the detector allows subsequent processing of the detected signals to infer spatial (e.g., depth localization, inverse modeling) information about the detected signals. Detectors of a module may be alternatively disposed on the module as may serve a particular implementation.

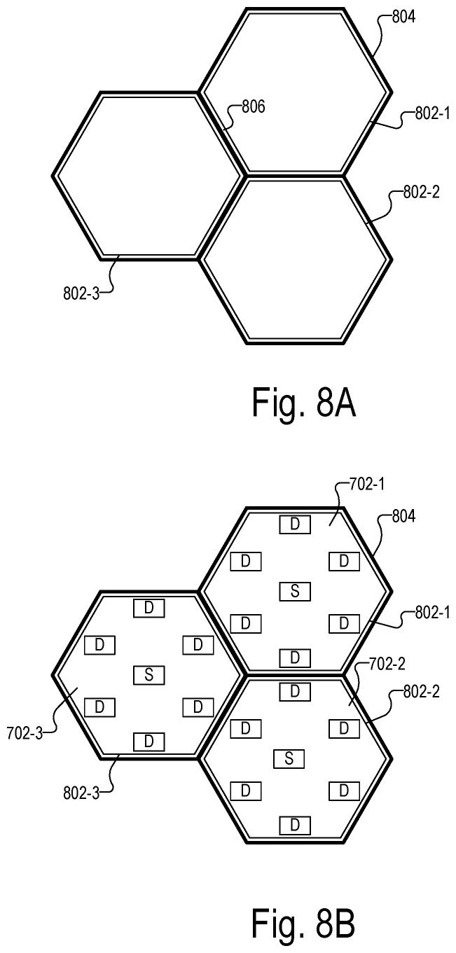

In FIG. 7, modules 702 are shown to be adjacent to and touching one another. Modules 702 may alternatively be spaced apart from one another. For example, FIGS. 8A-8B show an exemplary implementation of modular assembly 700 in which modules 702 are configured to be inserted into individual slots 802 (e.g., slots 802-1 through 802-3, also referred to as cutouts) of a wearable assembly 804. In particular, FIG. 8A shows the individual slots 802 of the wearable assembly 804 before modules 702 have been inserted into respective slots 802, and FIG. 8B shows wearable assembly 804 with individual modules 702 inserted into respective individual slots 802.

Wearable assembly 804 may implement wearable assembly 602 and may be configured as headgear and/or any other type of device configured to be worn by a user.

As shown in FIG. 8A, each slot 802 is surrounded by a wall (e.g., wall 806) such that when modules 702 are inserted into their respective individual slots 802, the walls physically separate modules 702 one from another. In alternative embodiments, a module (e.g., module 702-1) may be in at least partial physical contact with a neighboring module (e.g., module 702-2).

Each of the modules described herein may be inserted into appropriately shaped slots or cutouts of a wearable assembly, as described in connection with FIGS. 8A-8B. However, for ease of explanation, such wearable assemblies are not shown in the figures.

As shown in FIGS. 7 and 8B, modules 702 may have a hexagonal shape. Modules 702 may alternatively have any other suitable geometry (e.g., in the shape of a pentagon, octagon, square, rectangular, circular, triangular, free-form, etc.).

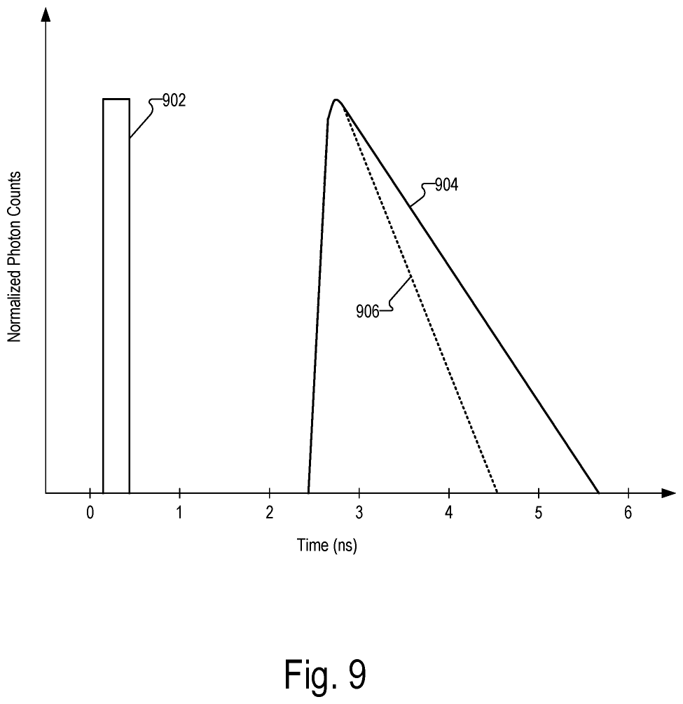

If a light signal emitted by a light source is relatively intense, a resultant histogram (e.g., TPSF) may be distorted. To illustrate, FIG. 9 shows a light pulse 902 followed by an ideal histogram 904 that occurs if the intensity of light pulse 902 is below a threshold. FIG. 9 also shows a distorted histogram 906 that may occur if the intensity of light pulse 902 is above the threshold. As shown, the tail portion of distorted histogram 906 is steeper in slope than the tail port of ideal histogram 904. This may be caused by photon pile up that occurs when there are relatively short light source-detector distances and/or when the absorption of tissue being measured changes. In neuroscience, a hemodynamic signal causes a change in the absorption properties of the tissue when there is neural activity. As a result, a distorted histogram 906 may result in inaccurate or ineffective measurements.

One approach for preventing distorted histograms includes using fast gating circuitry to ensure that a photodetector is turned off when it would be saturated (e.g., for a short time period subsequent to the occurrence of the light pulse, such as described in FIG. 3) and turned on after the possibility of saturation decreases or passes. Additionally this distortion can be avoided by using low laser intensities (e.g., that result in count rates that are limited to one to two percent) at the expense of measurement/acquisition time. Both of these approaches are suboptimal because gating requires extra circuit complexity and leads to higher power consumption whereas lowering the laser signal results in measurement times that are too slow to capture the desired hemodynamic signals.

However, in accordance with the systems, circuits, and methods described herein, because each detector includes many photodetectors in parallel and because each photodetector is independently controlled by a corresponding TDC, the dynamic range of the detector is increased and the effect of pile-up is mitigated. Through this parallelism, the optical measurement systems described herein are able to operate at relatively high laser intensities (e.g., that support count rates up to ten percent or more) and also eliminate the need for fast gating circuitry. This is because a signal of interest is evenly distributed across an array of photodetectors. Consequently, each photodetector included in the array is able to independently measure the same response.

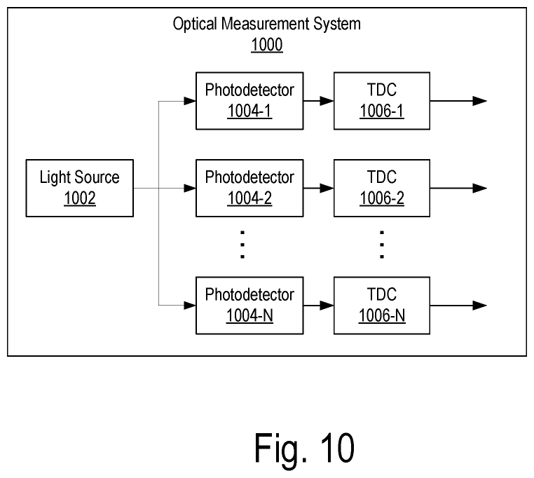

To illustrate this parallelism, FIG. 10 shows an exemplary optical measurement system 1000 that may be similar to and/or implement any of the optical measurement systems described herein. As shown, optical measurement system 1000 includes a light source 1002, a plurality of photodetectors 1004 (e.g., photodetectors 1004-1 through 1004-N), and a plurality of TDCs 1006 (e.g., TDCs 1006-1 through 1006-N). Each of these elements may be similar to the elements described herein. For example, light source 1002 may be implemented by a laser and/or any other suitable light source configured to emit light pulses (e.g., laser pulses). Photodetectors 1004 may be implemented by SPADs and may be configured to detect photons of the light pulses after the light pulses are scattered by the target. TDCs 1006 may be configured to record timestamp symbols representative of when the photons are detected by photodetectors 1004.

As illustrated in FIG. 10, optical measurement system 1000 may include N photodetectors 1004 and N TDCs 1006, where N is a relatively high number (e.g., greater than 64, such as 256, 512, . . . , 16384, etc.). Each TDC 1006 corresponds to and is configured to detect photon arrival times for a different photodetector 1004. For example, TDC 1006-1 corresponds to and is configured to detect photon arrival times for photodetector 1004-1, TDC 1006-2 corresponds to and is configured to detect photon arrival times for photodetector 1004-2, etc. Each photodetector-TDC pair is configured to detect, in parallel, photon arrival times for light pulses emitted by the same light source 1002.

As described herein, because optical measurement system 1000 includes so many photodetector-TDC pairs in parallel, the photodetectors do not need to be fast gated (e.g., armed and disarmed during a time subsequent to each light pulse emitted by light source 1002, as described in connection with FIG. 3). This may obviate the need for fast gating circuitry for each of the photodetectors 1004.

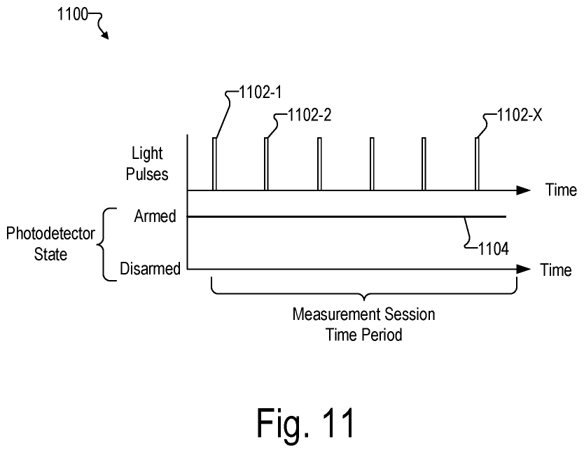

To illustrate, FIG. 11 illustrates an exemplary timing diagram 1100 for performing an optical measurement operation using optical measurement system 1000. As shown, light source 1002 may emit a sequence of light pulses 1102 (e.g., light pulses 1102-1 through 1102-X, where X is any suitable integer) during a measurement session time period. The measurement session time period may be of any suitable duration. As shown, light pulses 1102 are temporally spaced on from another within the measurement session time period by a plurality of time periods during which light source 1002 (FIG. 10) does not emit light.

As illustrated by line 1104, each photodetector 1004 (FIG. 10) is configured to remain in an armed state during an entire duration of the measurement session time period. While in the armed state, one or more of photodetectors 1004 may be configured to detect photons from light pulses 1102 (e.g., during the time periods that temporally separate light pulses 1102). Photodetectors 1004 may remain in the armed state during the measurement session time period in any suitable manner. For example, if there is no fast gating circuitry, photodetectors 1004 may remain in the armed state by receiving operating power from a component within optical measurement system 1000.

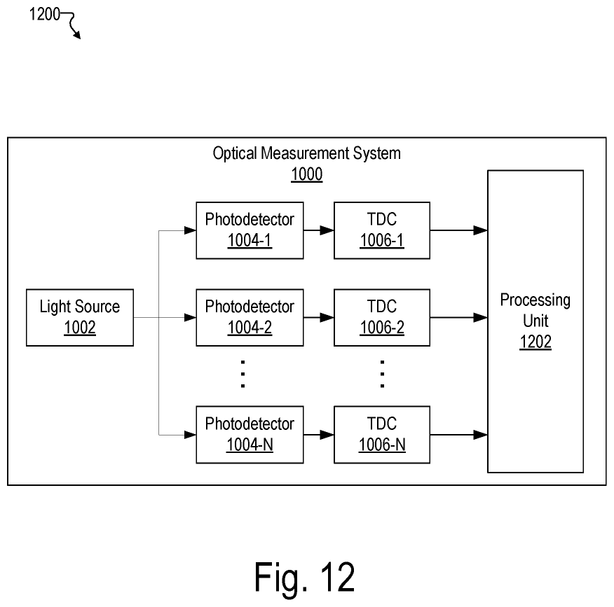

FIG. 12 shows an exemplary implementation 1200 of optical measurement system 1000 in which optical measurement system 1000 further includes a processing unit 1202. Processing unit 1202 is configured to receive output signals from TDCs 1006 and perform, based on the output signals, one or more operations. For example, as described herein, processing unit 1202 may be configured to generate one or more histograms based on the output signals. The output signals may include data representative of recorded timestamp symbols, as described herein.

Processing unit 1202 may be implemented by processor 108, controller 112, control circuit 204, shown in FIG. 1, and/or any other suitable processing and/or computing device or circuit.

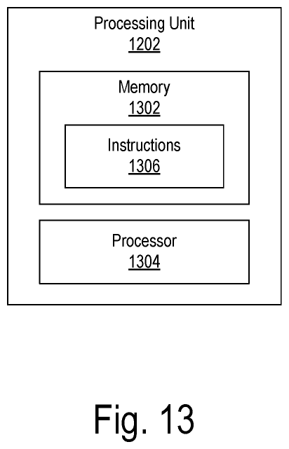

For example, FIG. 13 illustrates an exemplary implementation of processing unit 1202 in which processing unit 1202 includes a memory 1302 and a processor 1304 configured to be selectively and communicatively coupled to one another. In some examples, memory 1302 and processor 1304 may be distributed between multiple devices and/or multiple locations as may serve a particular implementation.

Memory 1302 may be implemented by any suitable non-transitory computer-readable medium and/or non-transitory processor-readable medium, such as any combination of non-volatile storage media and/or volatile storage media. Exemplary non-volatile storage media include, but are not limited to, read-only memory, flash memory, a solid-state drive, a magnetic storage device (e.g., a hard drive), ferroelectric random-access memory (“RAM”), and an optical disc. Exemplary volatile storage media include, but are not limited to, RAM (e.g., dynamic RAM).

Memory 1302 may maintain (e.g., store) executable data used by processor 1304 to perform one or more of the operations described herein. For example, memory 1302 may store instructions 1306 that may be executed by processor 1304 to perform any of the operations described herein. Instructions 1306 may be implemented by any suitable application, program (e.g., sound processing program), software, code, and/or other executable data instance. Memory 1302 may also maintain any data received, generated, managed, used, and/or transmitted by processor 1304.

Processor 1304 may be configured to perform (e.g., execute instructions 1306 stored in memory 1302 to perform) various operations described herein. For example, processor 1304 may be configured to perform any of the operations described herein as being performed by processing unit 1202.

In some examples, processing unit 1202 may be included in the same wearable assembly (e.g., wearable assembly 602) that include light source 1002, photodetectors 1004, and/or TDCs 1006. Alternatively, processing unit 1202 is not included in the same wearable assembly (e.g., wearable assembly 602) that include light source 1002, photodetectors 1004, and/or TDCs 1006.

To illustrate, processing unit 1202 may be included in a wearable device separate from wearable assembly 602. For example, processing unit 1202 may be included in a wearable device configured to be worn off the head while wearable assembly 602 is worn on the head. In these examples, one or more communication interfaces (e.g., cables, wireless interfaces, etc.) may be used to facilitate wearable assembly 602 and the separate wearable device.

Additionally or alternatively, processing unit 1202 may be remote from the user (i.e., not worn by the user). For example, processing unit 1202 may be implemented by a stand-alone computing device communicatively coupled to wearable assembly 602 by way of one or more communication interfaces (e.g., cables, wireless interfaces, etc.).

Processing unit 1202 may be configured to generate one or more histograms based on the output signals provided by TDCs 1006. For example, processing unit 1202 may generate a single histogram based on the output signals provided by all the TDCs 1006. Alternatively, processing unit 1202 may generate a plurality of histograms each based on a subset of the output signals provided by TDCs 1006.

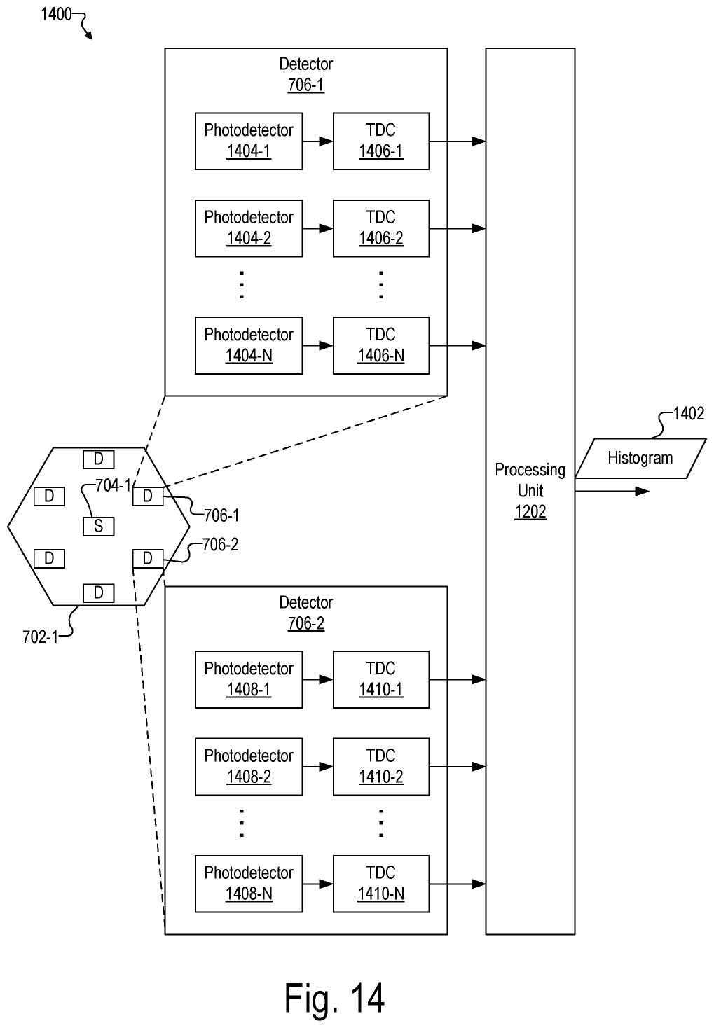

To illustrate, FIG. 14 shows an exemplary configuration 1400 in which processing unit 1202 is configured to generate a single histogram 1402 based on output signals provided by TDCs of multiple detectors of a module. In particular, FIG. 14 shows that detector 706-1 of module 702-1 includes a first plurality of photodetectors 1404 (e.g., photodetectors 1404-1 through 1404-N) and a first plurality of TDCs 1406 (e.g., TDCs 1406-1 through 1406-N) corresponding to photodetectors 1404. Likewise, FIG. 14 shows that detector 706-2 of module 702-1 includes a second plurality of photodetectors 1408 (e.g., photodetectors 1408-1 through 1408-N) and a second plurality of TDCs 1410 (e.g., TDCs 1410-1 through 1410-N) corresponding to photodetectors 1408.

Photodetectors 1404 and 1408 may operate in a similar manner to any of the other photodetectors described herein. Likewise, TDCs 1406 and 1410 may operate in a similar manner to any of the other TDCs described herein. For example, photodetectors 1404 and TDCs 1406 may together detect photon arrival times for photons of light pulses emitted by light source 704-1 (and/or a light source included in a different module). Likewise, photodetectors 1408 and TDCs 1410 may together detect photon arrival times for photons of light pulses emitted by light source 704-1 (and/or a light source included in a different module).

As shown, processing unit 1202 may be configured to receive the output signals provided by TDCs 1406 and 1410 and generate a single histogram based on the output signals. This may be performed in any suitable manner.

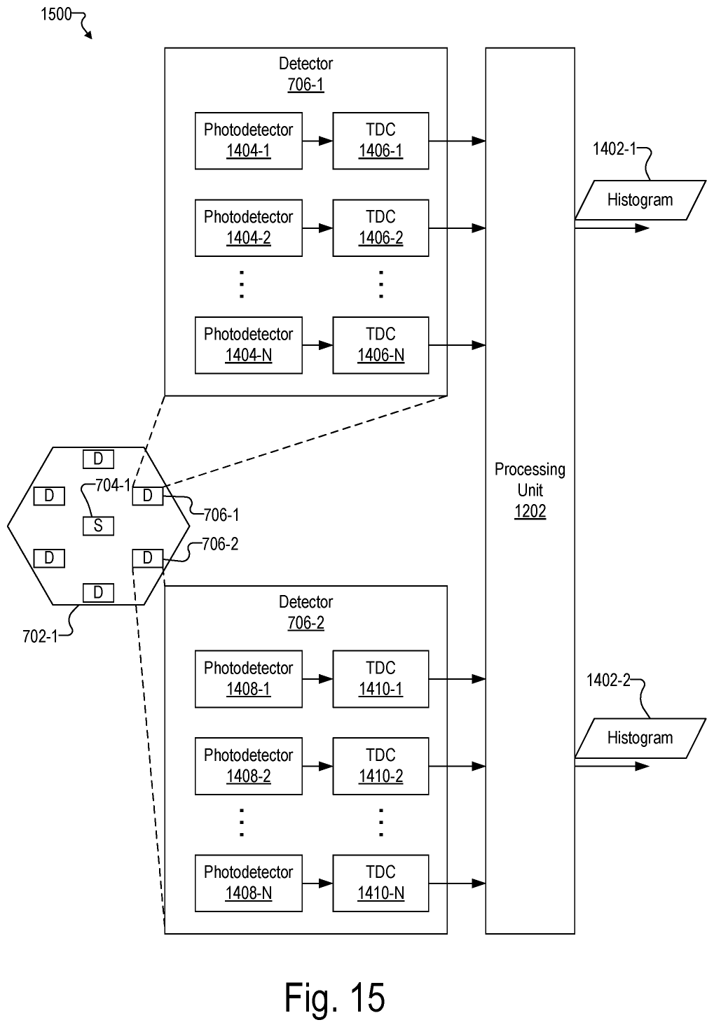

FIG. 15 shows an alternative configuration 1500 in which processing unit 1202 is configured to generate multiple histograms each based on a different subset of the output signals provided by TDCs 1406 and 1410. As shown, processing unit 1202 may be configured to generate a first histogram 1402-1 based on output signals provided by TDCs 1406 and a second histogram 1402-2 based on output signals provided by TDCs 1410.

Generating histograms based on these smaller subsets of TDC output signals may be beneficial in some applications where bandwidth and processing capabilities may be limited. In some examples, processing unit 1202 may combine histograms 1402-1 and 1402-2 into a single histogram (e.g., at a later time when more processing resources and/or time are available).

In some examples, histograms generated by processing unit 1202 may be based on output signals provided by TDCs in different modules. For example, processing unit 1202 may generate a first histogram based on output signals provided by TDCs included in module 702-1 and a second histogram based on output signals provided by TDCs included in module 702-2. In this example, the measurement session time periods associated with the first and second histograms may at least partially overlap. Alternatively, the measurement session time periods associated with the first and second histograms may be non-overlapping.

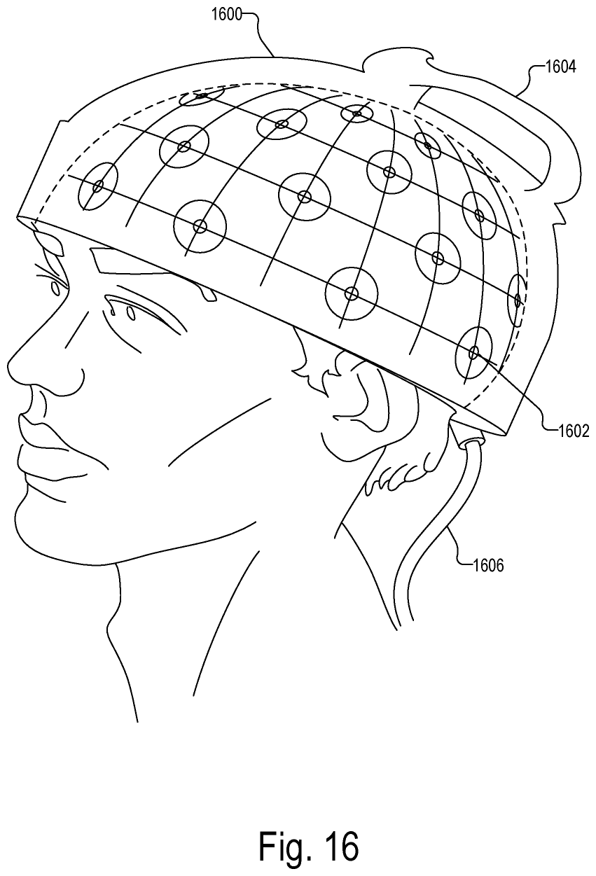

FIGS. 16-21 illustrate embodiments of a wearable device 1600 that includes elements of the optical detection systems described herein. In particular, the wearable devices 1600 shown in FIGS. 16-21 include a plurality of modules 1602, similar to the modules described herein. For example, each module 1602 may include a light source (e.g., light source 704-1) and a plurality of detectors (e.g., detectors 706-1 through 706-6). The wearable devices 1600 may each also include a controller (e.g., controller 112) and a processor (e.g., processor 108) and/or be communicatively connected to a controller and processor. In general, wearable device 1600 may be implemented by any suitable headgear and/or clothing article configured to be worn by a user. The headgear and/or clothing article may include batteries, cables, and/or other peripherals for the components of the optical measurement systems described herein.

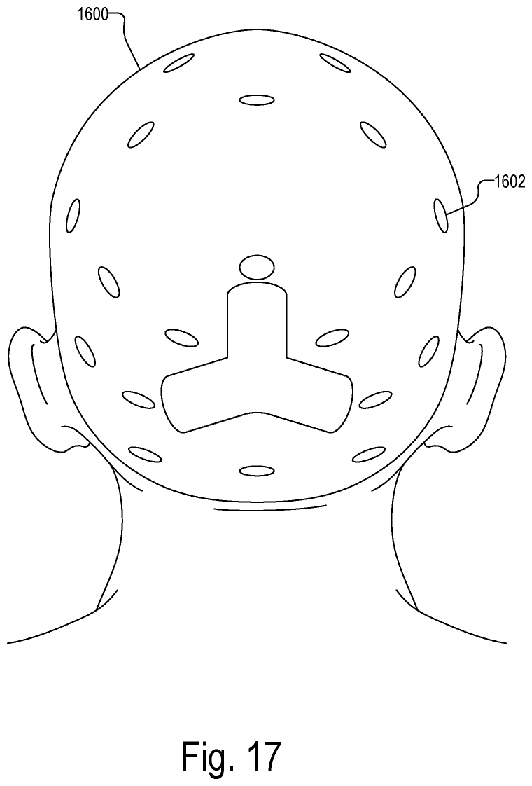

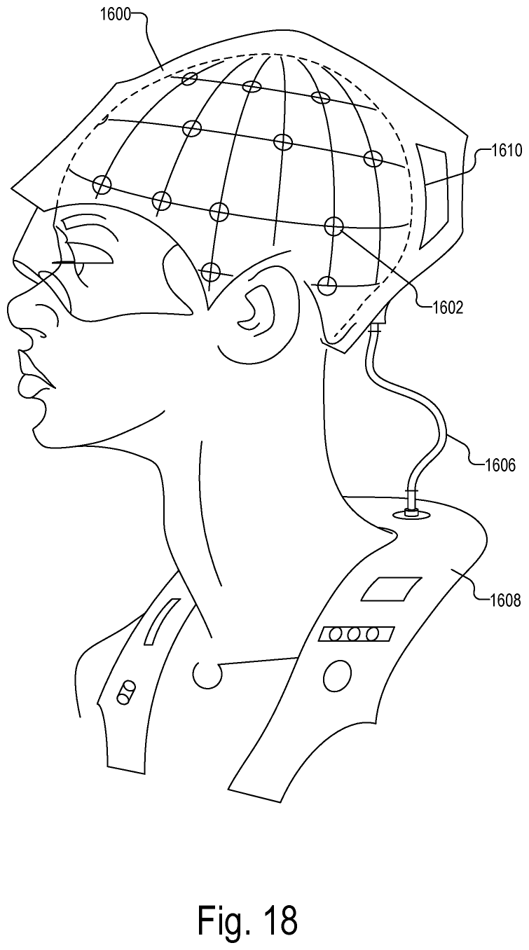

FIG. 16 illustrates an embodiment of a wearable device 1600 in the form of a helmet with a handle 1604. A cable 1606 extends from the wearable device 1600 for attachment to a battery or hub (with components such as a processor or the like). FIG. 17 illustrates another embodiment of a wearable device 1600 in the form of a helmet showing a back view. FIG. 18 illustrates a third embodiment of a wearable device 1600 in the form of a helmet with the cable 1606 leading to a wearable garment 1608 (such as a vest or partial vest) that can include a battery or a hub. Alternatively or additionally, the wearable device 1600 can include a crest 1610 or other protrusion for placement of the hub or battery.

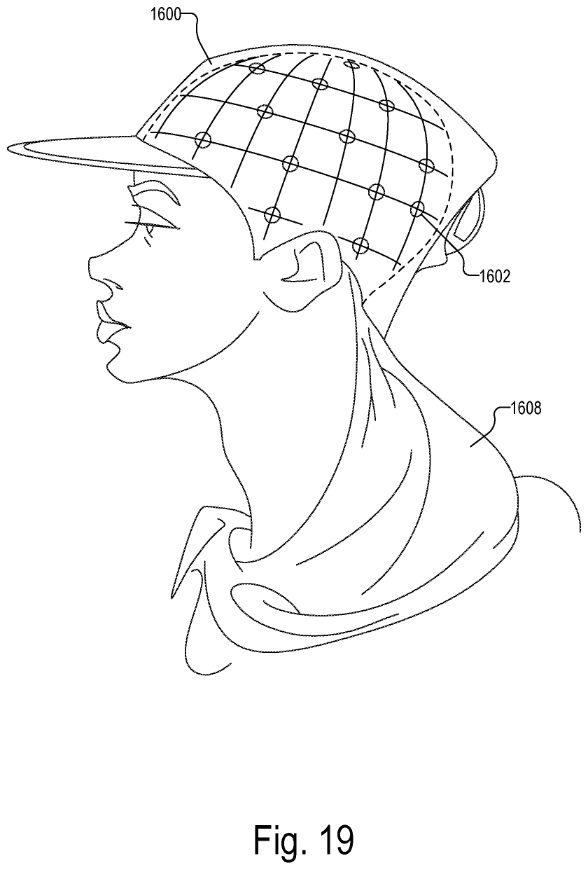

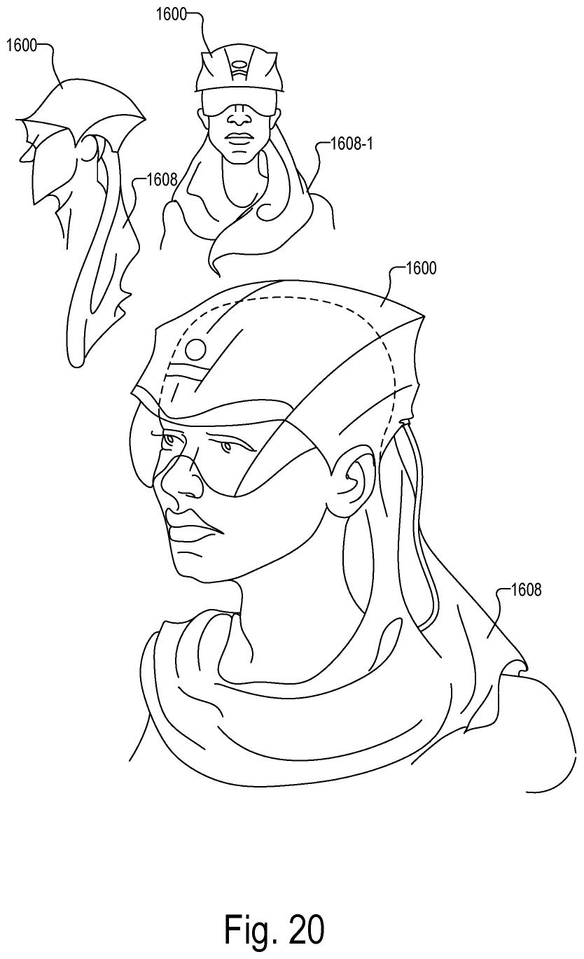

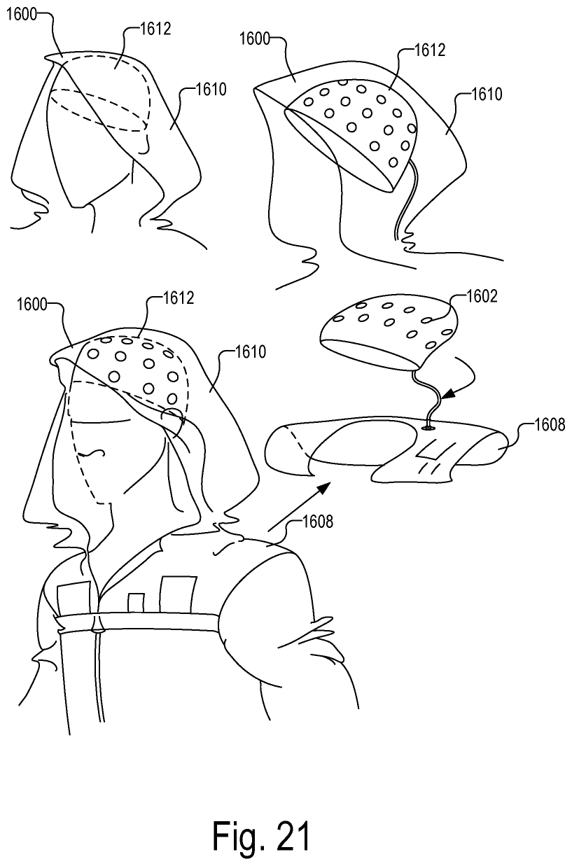

FIG. 19 illustrates another embodiment of a wearable device 1600 in the form of a cap with a wearable garment 1608 in the form of a scarf that may contain or conceal a cable, battery, and/or hub. FIG. 20 illustrates additional embodiments of a wearable device 1600 in the form of a helmet with a one-piece scarf 1608 or two-piece scarf 1608-1. FIG. 21 illustrates an embodiment of a wearable device 1600 that includes a hood 1610 and a beanie 1612 which contains the modules 1602, as well as a wearable garment 1608 that may contain a battery or hub.

In some examples, a non-transitory computer-readable medium storing computer-readable instructions may be provided in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Exemplary non-volatile storage media include, but are not limited to, read-only memory, flash memory, a solid-state drive, a magnetic storage device (e.g. a hard disk, a floppy disk, magnetic tape, etc.), ferroelectric random-access memory (“RAM”), and an optical disc (e.g., a compact disc, a digital video disc, a Blu-ray disc, etc.). Exemplary volatile storage media include, but are not limited to, RAM (e.g., dynamic RAM).

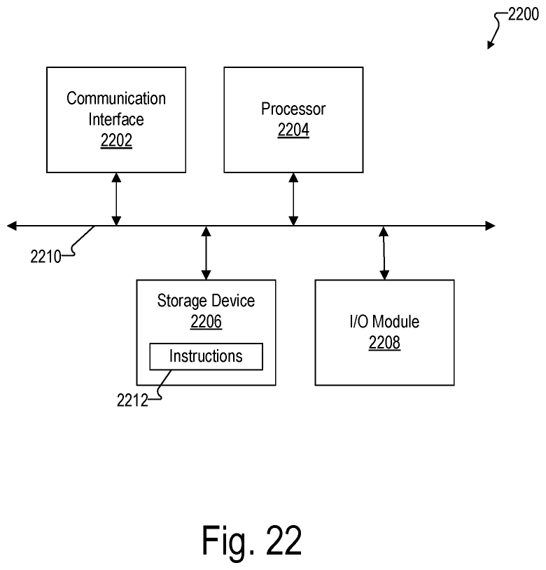

FIG. 22 illustrates an exemplary computing device 2200 that may be specifically configured to perform one or more of the processes described herein. Any of the systems, units, computing devices, and/or other components described herein may be implemented by computing device 2200.

As shown in FIG. 22, computing device 2200 may include a communication interface 2202, a processor 2204, a storage device 2206, and an input/output (“I/O”) module 2208 communicatively connected one to another via a communication infrastructure 2210. While an exemplary computing device 2200 is shown in FIG. 22, the components illustrated in FIG. 22 are not intended to be limiting. Additional or alternative components may be used in other embodiments. Components of computing device 2200 shown in FIG. 22 will now be described in additional detail.

Communication interface 2202 may be configured to communicate with one or more computing devices. Examples of communication interface 2202 include, without limitation, a wired network interface (such as a network interface card), a wireless network interface (such as a wireless network interface card), a modem, an audio/video connection, and any other suitable interface.

Processor 2204 generally represents any type or form of processing unit capable of processing data and/or interpreting, executing, and/or directing execution of one or more of the instructions, processes, and/or operations described herein. Processor 2204 may perform operations by executing computer-executable instructions 2212 (e.g., an application, software, code, and/or other executable data instance) stored in storage device 2206.

Storage device 2206 may include one or more data storage media, devices, or configurations and may employ any type, form, and combination of data storage media and/or device. For example, storage device 2206 may include, but is not limited to, any combination of the non-volatile media and/or volatile media described herein. Electronic data, including data described herein, may be temporarily and/or permanently stored in storage device 2206. For example, data representative of computer-executable instructions 2212 configured to direct processor 2204 to perform any of the operations described herein may be stored within storage device 2206. In some examples, data may be arranged in one or more databases residing within storage device 2206.

I/O module 2208 may include one or more I/O modules configured to receive user input and provide user output. I/O module 2208 may include any hardware, firmware, software, or combination thereof supportive of input and output capabilities. For example, I/O module 2208 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 2208 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 2208 is configured to provide graphical data to a display for presentation to a user. The graphical data may be representative of one or more graphical user interfaces and/or any other graphical content as may serve a particular implementation.

An illustrative optical measurement system includes a light source configured to emit, during a measurement session time period, a sequence of light pulses towards a target; a plurality of photodetectors configured to remain in an armed state during an entire duration of the measurement session time period, and detect, while in the armed state, photons of the light pulses after the light pulses are scattered by the target; and a plurality of TDCs comprising a different TDC for each photodetector of the plurality of photodetectors, the TDCs configured to record timestamp symbols representative of when the photons are detected by the photodetectors.

Another illustrative optical measurement system includes a wearable assembly configured to be worn by a user and including a first module. The first module includes 1) a first light source configured to emit, during a first measurement session time period, a first sequence of light pulses towards a target, 2) a first detector comprising a first plurality of photodetectors configured to remain in an armed state during an entire duration of the first measurement session time period, and detect, while in the armed state, photons of the first sequence of light pulses after the light pulses are scattered by the target, and 3) a second detector comprising a second plurality of photodetectors configured to remain in the armed state during the entire duration of the first measurement session time period, and detect, while in the armed state, photons of the first sequence of light pulses after the light pulses are scattered by the target.

Another illustrative optical measurement system includes a wearable assembly configured to be worn by a user and comprising a light source configured to emit, during a measurement session time period, a sequence of light pulses towards a target; a plurality of photodetectors configured to remain in an armed state during an entire duration of the measurement session time period, and detect, while in the armed state, photons of the light pulses after the light pulses are scattered by the target; a plurality of TDCs comprising a different TDC for each photodetector of the plurality of photodetectors, the TDCs configured to record timestamp symbols representative of when the photons are detected by the photodetectors; and a processing unit configured to perform an operation based on the recorded timestamp symbols.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. An optical measurement system comprising:
a light source configured to emit, during a measurement session time period, a sequence of light pulses towards a target;
a plurality of photodetectors configured to
remain in an armed state during an entire duration of the measurement session time period, and
detect, while in the armed state, photons of the light pulses after the light pulses are scattered by the target; and
a plurality of time-to-digital converters (TDCs) comprising a different TDC for each photodetector of the plurality of photodetectors, the TDCs configured to record timestamp symbols representative of when the photons are detected by the photodetectors.

2. The optical measurement system of claim 1, further comprising:
a module comprising a plurality of detectors;
wherein the photodetectors and the TDCs are all included in a particular detector included in the plurality of detectors.

3. The optical measurement system of claim 2, wherein the light source is included in the particular detector.

4. The optical measurement system of claim 2, where the light source is included in an additional detector included in the plurality of detectors.

5. The optical measurement system of claim 2, further comprising:
a wearable assembly configured to be worn by a user;
wherein the module is configured to be attached to the wearable assembly.

6. The optical measurement system of claim 5, further comprising one or more additional modules each including photodetectors and TDCs and each configured to be attached to the wearable assembly.

7. The optical measurement system of claim 5, wherein the wearable assembly comprises headgear configured to be worn on a head of the user.

8. The optical measurement system of claim 2, further comprising:
a second plurality of photodetectors included in an additional detector included in the plurality of detectors, the second plurality of photodetectors configured to:
remain in the armed state during the entire duration of the measurement session time period, and
detect, while in the armed state, photons of the light pulses after the light pulses are scattered by the target; and
a second plurality of TDCs included in the additional detector, the second plurality of TDCs comprising a different TDC for each photodetector of the second plurality of photodetectors, the second plurality of TDCs configured to record timestamp symbols representative of when the photons are detected by the second plurality of photodetectors.

9. The optical measurement system of claim 1, further comprising a processing unit configured to:
receive output signals from the TDCs; and
perform an operation based on the output signals.

10. The optical measurement system of claim 9, wherein the performing of the operation comprises generating a single histogram based on the output signals.

11. The optical measurement system of claim 9, wherein the performing of the operation comprises:
generating a first histogram based on a first subset of the output signals; and
generating a second histogram based on a second subset of the output signals.

12. The optical measurement system of claim 11, wherein the processing unit is configured to combine the first and second histograms into a single histogram.

13. The optical measurement system of claim 1, wherein the optical measurement system does not include fast gating circuitry configured to arm and disarm the photodetectors.

14. The optical measurement system of claim 1, wherein the plurality of photodetectors comprises a plurality of single-photon avalanche diodes (SPADs).

15. An optical measurement system comprising:
a wearable assembly configured to be worn by a user and comprising:
a first module comprising:
a first light source configured to emit, during a first measurement session time period, a first sequence of light pulses towards a target;
a first detector comprising:
a first plurality of photodetectors configured to
remain in an armed state during an entire duration of the first measurement session time period, and
detect, while in the armed state, photons of the first sequence of light pulses after the light pulses are scattered by the target; and
a second detector comprising:
a second plurality of photodetectors configured to
remain in the armed state during the entire duration of the first measurement session time period, and
detect, while in the armed state, photons of the first sequence of light pulses after the light pulses are scattered by the target.

16. The optical measurement system of claim 15, wherein:
the first detector further comprises a first plurality of time-to-digital converters (TDCs) comprising a different TDC for each photodetector of the first plurality of photodetectors, the first plurality of TDCs configured to record timestamp symbols representative of when the photons are detected by the first plurality of photodetectors; and
the second detector further comprises a second plurality of TDCs comprising a different TDC for each photodetector of the second plurality of photodetectors, the second plurality of TDCs configured to record timestamp symbols representative of when the photons are detected by the second plurality of photodetectors.

17. The optical measurement system of claim 16, further comprising a processing unit configured to:
receive a first plurality of output signals from the first plurality of TDCs and a second plurality of output signals from the second plurality of TDCs; and
perform an operation based on the first and second pluralities of output signals.

18. The optical measurement system of claim 17, wherein the performing of the operation comprises generating a first histogram based on the first plurality of output signals and a second histogram based on the second plurality of output signals.

19. The optical measurement system of claim 17, wherein the performing of the operation comprises:
generating a first histogram based on a first subset of the first plurality of output signals; and
generating a second histogram based on a second subset of the first plurality of output signals.

20. The optical measurement system of claim 19, wherein the processing unit is configured to combine the first and second histograms into a single histogram.

21. The optical measurement system of claim 17, wherein the processing unit is included in the wearable assembly.

22. The optical measurement system of claim 17, wherein the processing unit is not included in the wearable assembly.

23. The optical measurement system of claim 15, wherein the optical measurement system does not include fast gating circuitry configured to arm and disarm the first and second pluralities of photodetectors.

24. The optical measurement system of claim 15, wherein the first and second pluralities of photodetectors comprise a plurality of single-photon avalanche diodes (SPADs).

25. The optical measurement system of claim 15, wherein the wearable assembly further comprises:
a second module comprising:
a second light source configured to emit, during a second measurement session time period, a second sequence of light pulses towards a target;
a third detector comprising:
a third plurality of photodetectors configured to
remain in an armed state during an entire duration of the second measurement session time period, and
detect, while in the armed state, photons of the second sequence of light pulses after the light pulses are scattered by the target; and
a fourth detector comprising:
a fourth plurality of photodetectors configured to
remain in the armed state during the entire duration of the second measurement session time period, and
detect, while in the armed state, photons of the second sequence of light pulses after the light pulses are scattered by the target.

26. The optical measurement system of claim 25, wherein the first and second measurement session time periods at least partially overlap.

27. The optical measurement system of claim 25, wherein the first and second measurement session time periods are non-overlapping.

28-35. (canceled)
